(19) United States
(12) Patent Application Publication
(10) Pub. No.: US 2021/0158968 A1
(43) Pub. Date: May 27, 2021

(54) ANIMAL HEALTH RISK EVALUATION SYSTEM AND ANIMAL HEALTH RISK EVALUATION METHOD

(71) Applicant: FUJIFILM Corporation, Tokyo (JP)

(72) Inventors: Takeshi FUKUDA, Ashigarakami-gun (JP); Haruyasu NAKATSUGAWA, Ashigarakami-gun (JP)

(73) Assignee: FUJIFILM Corporation, Tokyo (JP)

(21) Appl. No.: 17/098,663

(22) Filed: Nov. 16, 2020

(30) Foreign Application Priority Data
Nov. 25, 2019 (JP) 2019-212496

(51) Int. Cl. G16H 50/30; G16H 10/60; G16H 50/70; G16H 40/67; G16H 10/40; G16H 50/50; G06Q 40/08; G06Q 10/10; G16H 40/20; A61B 5/00; A61B 6/00; A61B 10/00; A61B 5/055; A61B 6/03; A61B 8/08

(52) U.S. Cl. CPC G16H 50/30; A61B 2503/40; G16H 50/70; G16H 40/67; G16H 10/40; G16H 50/50; G06Q 40/08; G06Q 10/10; G16H 40/20; A61B 5/7275; A61B 5/4842; A61B 6/503; A61B 10/0038; A61B 5/055; A61B 6/032; A61B 8/5223; A61B 5/7264; A61B 6/5217; G16H 10/60

(57) ABSTRACT

An animal health risk evaluation system includes an examination value acquisition unit that acquires an examination value as a result of an examination executed on an animal, a breed data acquisition unit that acquires breed data as information for specifying a breed of the animal, a storage unit that stores the breed data in association with the examination value, and a risk evaluation unit that evaluates a future risk pertaining to health of a specific animal as an evaluation target using the breed data and the examination value of the specific animal.

### CROSS-REFERENCE TO RELATED APPLICATIONS

This application claims priority under 35 U.S.C § 119(a) to Japanese Patent Application No. 2019-212496 filed on 25 Nov. 2019. The above application is hereby expressly incorporated by reference, in its entirety, into the present application.

### BACKGROUND OF THE INVENTION

### 1. Field of the Invention

The present invention relates to a system for evaluating a risk pertaining to health of a pet animal or the like and an animal health risk evaluation method.

### 2. Description of the Related Art

Hitherto, there is a case where an expense (hereinafter, referred to as a medical expense) pertaining to medical care, treatment, or the like for illness or injury is expensive. Furthermore, there is a concern that an expensive medical expense that is suddenly required imperils the normal life. For this reason, a human subscribes to any medical insurance other than the National Health Insurance, and reduces the risk pertaining to medical care described above.

Since a pet animal (so-called pet) or a domestic animal is also an animal like a human, the animal may of course suffer illness or injury, and treatment or the like is required. For this reason, in order to prepare for a medical expense pertaining to illness or injury of an animal, a keeper of the animal may subscribe to an animal medical insurance. Furthermore, in recent years, there is known an application that, in a case where a result of a urine examination of a pet is input, performs an introduction of a hospital having a medical specialist, provision of information relating to an insurance premium, provision of information relating to an insurance plan, or the like according to a degree of health calculated based on a result of a urine examination (JP2018-156123A).

### SUMMARY OF THE INVENTION

In comparison with a human medical insurance, a plan and an insurance premium of an animal medical insurance are simple, and an animal as an affected animal and a keeper of the animal may not sufficiently reduce a risk pertaining to medical care.

For example, for subscription to an animal medical insurance, provision of information, such as animal species (dog, cat, or the like), breed (Golden Retriever, American Shorthair, or the like), age, and sex, to an insurance company is required; however, an insurance premium of a prepared insurance plan is undifferentiated without depending on an animal species, a breed, and the like. Furthermore, for example, there are only two kinds of plans including a plan where a keeper is charged 50% of a medical expense and a plan where the keeper is charged 70% of the medical expense, regardless of a breed or the like of an animal as a target of insurance. Accordingly, an insurance premium may not be worth a risk depending on the breed or the like. In addition, in a case where there is a past medical history, insurance subscription may be rejected. In this case, even though an animal suffers illness or injury to be treated, the animal may not receive appropriate care.

One of causes of the problem of the animal medical insurance described above is that there are various kinds of animals, and accordingly, risk evaluation pertaining to health necessary to individually set an appropriate insurance plan for each kind is not appropriately performed.

An object of the invention is to provide an animal health risk evaluation system and an animal health risk evaluation method capable of appropriately evaluating a risk pertaining to health of an animal.

An animal health risk evaluation system according to an aspect of the invention comprises an examination value acquisition unit that acquires an examination value as a result of an examination executed on an animal, a breed data acquisition unit that acquires breed data as information for specifying a breed of the animal, a storage unit that stores the breed data in association with the examination value, and a risk evaluation unit that evaluates a future risk pertaining to health of a specific animal as an evaluation target using the breed data and the examination value of the specific animal.

It is preferable that the examination value acquisition unit acquires the examination value directly or indirectly from examination devices provided in one or a plurality of animal hospitals.

It is preferable that the examination value acquisition unit acquires one or a plurality of the examination values including a result of a blood examination.

It is preferable that the risk evaluation unit evaluates, as the risk, at least one of a probability that the specific animal is affected with a specific disease, a degree of seriousness in a case where the specific animal is affected with the specific disease, a lifetime, or a medical expense.

It is preferable that the risk evaluation unit evaluates the risk when a specific time elapses from a present time at which evaluation is performed.

It is preferable that the risk evaluation unit evaluates the risk by predicting transition of the examination value.

It is preferable that the risk evaluation unit evaluates the risk based on at least one of deviation of the examination value from a normal value range or a deviation amount of the examination value from the normal value range.

It is preferable that the animal health risk evaluation system further comprises an age data acquisition unit that acquires age data of the animal, the storage unit stores the age data in association with the breed data and the examination value, and the risk evaluation unit evaluates the risk of the specific animal by referring to the breed data, the examination value, and the age data stored in the storage unit.

It is preferable that the animal health risk evaluation system further comprises a risk evaluation value provision unit that provides an evaluation value of the risk evaluated by the risk evaluation unit to an insurance company.

It is preferable that the animal health risk evaluation system further comprises a special contract information acquisition unit that, in a case where the insurance company that receives the evaluation value of the risk provides a special contract on an animal medical insurance, acquires information pertaining to the special contract.

It is preferable that the animal health risk evaluation system further comprises a special contract information provision unit that provides the information pertaining to the special contract to an animal hospital.

An animal health risk evaluation method according to another aspect of the invention causes a computer to execute processing of reading an examination value as a result of an examination executed on an animal and breed data as information for specifying a breed of the animal from a database, which stores the examination value in association with the breed data, and evaluating a future risk pertaining to health of a specific animal as an evaluation target using the examination value and the breed data read from the database, and the breed data and the examination value of the specific animal.

It is preferable that the animal health risk evaluation method causes the computer to execute processing of presenting at least one of an insurance plan or a special contract based on the risk.

The animal health risk evaluation system and the animal health risk evaluation method according to the aspects of the invention can appropriately evaluate a risk pertaining to health of an animal.

### BRIEF DESCRIPTION OF THE DRAWINGS

FIG. 1 is an explanatory view showing a relationship between an animal health risk evaluation system and an animal hospital, and an insurance company.

FIG. 2 is a block diagram showing an example of examination devices in the animal hospital.

FIG. 3 is a block diagram showing the configuration of the animal health risk evaluation system.

FIG. 4 is a table showing an example of animal species and breeds.

FIG. 5 is a table showing a relationship between a breed of an aged dog at 13 years old or more and a ground for insurance claim.

FIG. 6 is a table showing a relationship between a disease or a symptom and a medical expense.

FIG. 7 is a table showing a relationship between an animal species and an average medical expense.

FIG. 8 is a flowchart showing the operation of the animal health risk evaluation system.

FIG. 9 is a graph showing an example of a method of risk evaluation.

FIG. 10 is a table showing a ground for insurance claim of Toy Poodle by age group.

FIG. 11 is a table showing a ground for insurance claim of Shiba Inu by age group.

FIG. 12 is a block diagram showing the configuration of an animal health risk evaluation system of a second embodiment.

FIG. 13 is a block diagram showing the configuration of an animal health risk evaluation system of a third embodiment.

FIG. 14 is a block diagram showing the configuration of an animal health risk evaluation system of a modification example of the third embodiment.

FIG. 15 is a block diagram showing the configuration of an animal health risk evaluation system of a fourth embodiment.

FIG. 16 is an explanatory view showing a learned model that functions as a risk evaluation unit.

FIG. 17 is an explanatory view showing a learned model that functions as a risk evaluation unit in the animal health risk evaluation system of the second embodiment.

FIG. 18 is an explanatory view showing a learned model that functions as a risk evaluation unit in the animal health risk evaluation system of the fourth embodiment.

### DESCRIPTION OF THE PREFERRED EMBODIMENTS

### First Embodiment

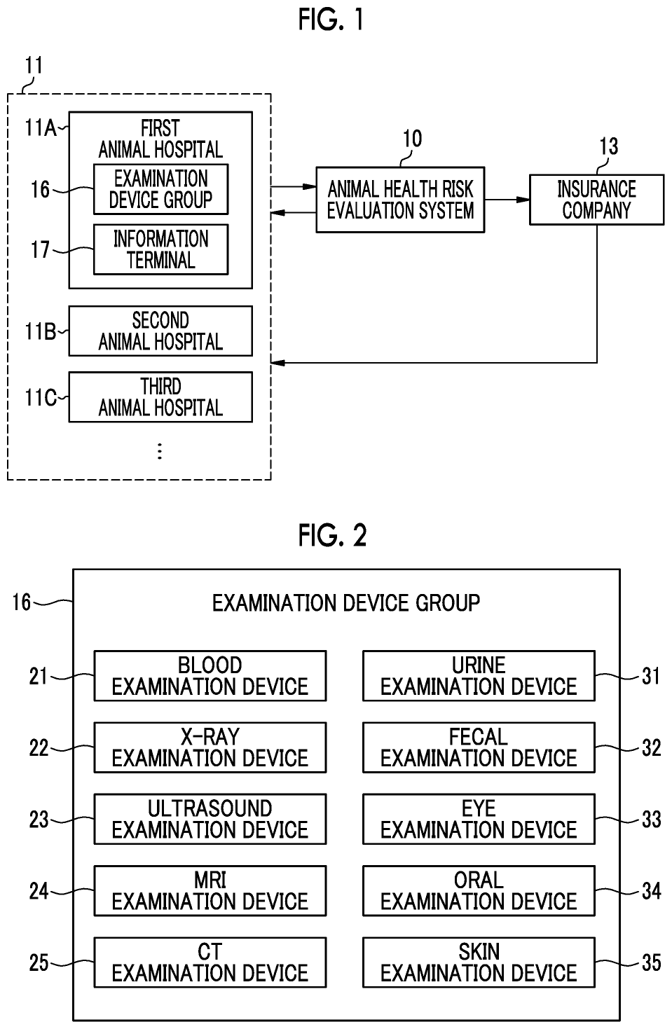

As shown in FIG. 1, an animal health risk evaluation system 10 is a system that cooperates with an animal hospital 11 to evaluate a future risk (hereinafter, referred to as a health risk) pertaining to health of an animal as an affected animal. The animal hospital 11 with which the animal health risk evaluation system 10 cooperates can include a plurality of animal hospitals, such as a first animal hospital 11A, a second animal hospital 11B, and a third animal hospital 11C. That is, the animal health risk evaluation system 10 can cooperate with one or a plurality of animal hospitals. In the embodiment, the animal health risk evaluation system 10 cooperates with a plurality of animal hospitals, such as the first animal hospital 11A, the second animal hospital 11B, and the third animal hospital 11C.

The cooperation of the animal health risk evaluation system 10 and the animal hospitals 11 means acquisition or provision of at least one of a result of diagnosis by a veterinarian, a result of an examination, an evaluation result of a health risk, or other kinds of information. Although the acquisition and provision of information can be performed by the animal health risk evaluation system 10 and the animal hospitals 11 in two directions, for example, the cooperation includes provision or acquisition of information in one direction, such as provision of an examination value and the like from the animal hospitals 11 to the animal health risk evaluation system 10 in one direction.

In the embodiment, the animal health risk evaluation system 10 receives information, which is used for evaluation of a health risk, from the animal hospitals 11 and provides information regarding the evaluation of the health risk, or the like to an insurance company 13. With this, the animal health risk evaluation system 10 prompts setting and provision of a wide variety of appropriate animal medical insurance plans.

Each of the animal hospitals 11 (the first animal hospital 11A, the second animal hospital 11B, the third animal hospital 11C, and the like) that cooperates with the animal health risk evaluation system 10 comprises an examination device group 16 and an information terminal 17.

As shown in FIG. 2, the examination device group 16 of the first animal hospital 11A has, for example, at least one of a blood examination device 21, an X-ray examination device 22, an ultrasound examination device 23, a magnetic resonance imaging (MRI) examination device 24, a computed tomography (CT) examination device 25, a urine examination device 31, a fecal examination device 32, an eye examination device 33, an oral examination device 34, a skin examination device 35, or the like. The veterinarian decides the types of necessary examinations, items, and the like in consideration of a symptom and the like and executes examination using various examination devices described above. In particular, there is a case where a specific examination (for example, blood examination) that can be used to distinguish many diseases is executed without depending on the symptom and the like.

The animal health risk evaluation system 10 can acquire examination values obtained by examinations using various examination devices described above from various examination devices directly or through the information terminal 17. The types and the number of specific examination devices configuring the examination device group 16 may be different depending on the scale or the like of the animal hospital 11. Note that, for cooperation with the animal health risk evaluation system 10, the examination device group 16 includes at least one examination device related to the evaluation of the health risk in the animal health risk evaluation system 10. In the embodiment, the animal health risk evaluation system 10 uses a result of a blood examination for the evaluation of the health risk.

The information terminal 17 is a so-called computer, and comprises a display unit (not shown), such as a liquid crystal display, and an operating unit (not shown) having a keyboard, a pointing device, and the like. The information terminal 17 is a device that manages medical charts and other kinds of information, and manages, for example, information regarding at least one of an identification number (ID), a name, a name of a keeper (owner), an animal species, a breed, an age, results of various examinations, findings, diagnosis results, or the like of an animal that becomes an affected animal. The information terminal 17 can acquire the examination value and the like automatically or by a manual input, and displays necessary information on the display unit.

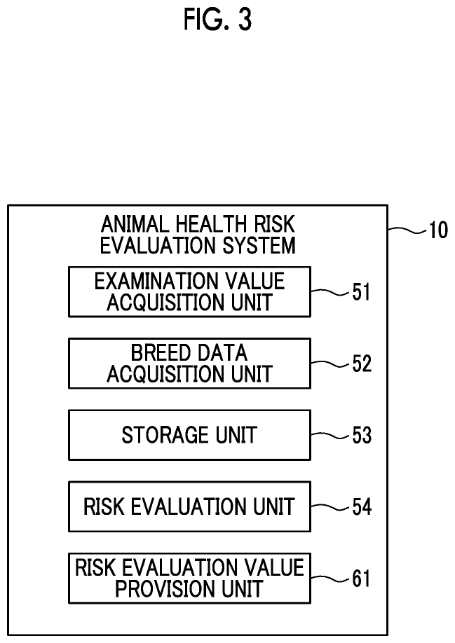

As shown in FIG. 3, the animal health risk evaluation system 10 comprises an examination value acquisition unit 51, a breed data acquisition unit 52, a storage unit 53, a risk evaluation unit 54, a risk evaluation value provision unit 61, and the like.

The examination value acquisition unit 51 acquires an examination value as a result of an examination executed on an animal. The examination value is the result of the examination and is a numerical value or the like in a form capable of being used for the evaluation of a health risk. For example, at least one of measurement values (the content of a predetermined component included in blood, and the like) directly obtained by various examination devices described above, a numerical value (a vertebral heart size (VHS) and the like) indicating the size of an organ or the like measured in an image examination of an X-ray image, a numerical value (a flag representing evaluation, such as the presence or absence of a disease or a progression degree) representing evaluation of the veterinarian based on the measurement values and the numerical values, or the like is used.

The examination value acquisition unit 51 acquires examination values from the examination devices provided in one or a plurality of animal hospitals 11 directly or indirectly. The direct acquisition of the examination values means that connection to the blood examination device 21 or the like is made to obtain an examination value of a blood examination result or the like instantly from the examination device. Furthermore, the indirect acquisition of the examination values means that connection to the information terminal 17 is made to acquire an examination value in the information terminal 17 or acquiring an examination value by an input operation on the examination value acquisition unit 51 using the information terminal 17.

The examination value acquired by the examination value acquisition unit 51 is an examination value that is used for evaluation of a health risk. Note that the examination value acquisition unit 51 can also acquire an examination value that is not used for the evaluation of the health risk in a case where the examination value is present. For example, even though only the result of the blood examination can evaluate a health risk relating to a specific disease, an examination value other than the result of the blood examination may be required in evaluating a health risk relating to another disease. For this reason, the examination value acquisition unit 51 can collect an examination value, which is not directly used for the evaluation of the health risk, as necessary. In the embodiment, at least the result of the blood examination is used for the evaluation of the health risk. For this reason, the examination value acquisition unit 51 acquires one or a plurality of examination values including the result of the blood examination. This is because the result of the blood examination becomes a determination material of the presence or absence of a disease and the degree of disease pertaining to comparatively many parts (kidney, heart, and the like), and actually, the veterinarian substantially always executes the blood examination; thus, the result of the blood examination is easily used for the evaluation of the health risk.

The examination values on the result of the blood examination include values, evaluation based on comparison with reference values or reference ranges for determining a range of normality and abnormality, and the like of total protein (TP), albumin (Alb), globulin (Glob), an albumin-globulin ratio, aspartate aminotransferase (AST), alanine aminotransferase (ALT), alkaline phosphatase (ALP), gamma glutamyl transpeptidase (GGT), total cholesterol (TCho), triglyceride (TG), total bilirubin (TBil), glucose (Glu), amylase (Amy), lipase (Lip), urea nitrogen (BUN), creatinine (Cre), a BUN-Cre ratio, symmetric dimethylarginine (SDMA), phosphorus (P), Ca (calcium), sodium (Na), potassium (K), chloride (Cl), and the like.

The breed data acquisition unit 52 acquires breed data as information for specifying a breed of the animal. The breed data is data including information pertaining to the breed, and is not only a name representing the breed but also information capable of specifying the breed directly or indirectly, such as a number or a symbol associated with the breed. In a case where information pertaining to the breed is acquired in a form hard to use for the evaluation of the health risk, such as a case where the name representing the breed is acquired as text, the breed data acquisition unit 52 performs conversion or the like of information into, for example, a numerical value associated with the breed on an one-to-one basis. In the embodiment, although the breed data acquisition unit 52 acquires the breed data through the information terminal 17, the breed data can be acquired from various examination devices of the examination device group 16, a management device of the examination device group 16, or the like, instead of the information terminal 17.

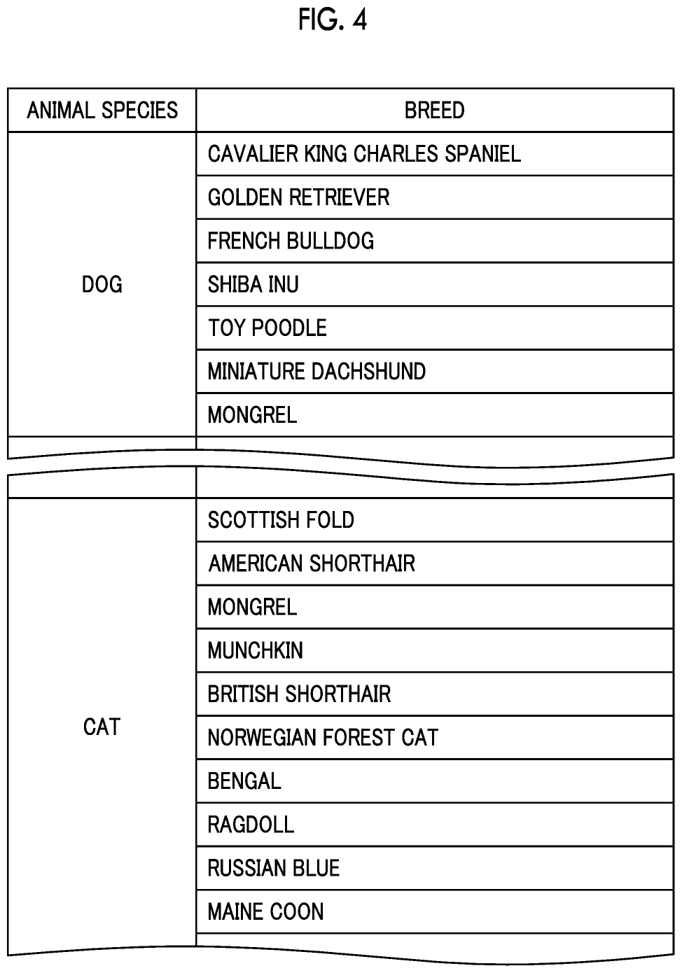

As shown in FIG. 4, the breed is a classification for subdividing a specific animal species in consideration of a genetic character or the like. For example, a classification for distinguishing between different species of intersterile animals, such as dog and cat, is an animal species. Then, for example, in regard to dog, Cavalier King Charles Spaniel, Golden Retriever, French Bulldog, and the like are breeds. Furthermore, the breeds include a “mongrel” made by mixing different breeds (mixed, hybrid, crossbred, or subspecies). Since the animal species and the breed have a relationship that the animal species is specified in a case where the breed is specified, the breed information includes information pertaining to the animal species, and also functions as information for specifying the animal species.

The storage unit 53 is a database pertaining to diseases, symptoms, and the like of animals, and stores at least the breed data and the examination values in association with each other. In the embodiment, the storage unit 53 stores the breed data, the examination values of various examinations, age, diseases or symptoms, the progress of diseases or symptoms, expenses pertaining to treatment, and the like in association with one another.

The risk evaluation unit 54 evaluates a health risk of a specific animal as an evaluation target using breed data and examination values of the specific animal. The specific animal as the evaluation target is an affected animal that visits the animal hospital 11. The risk evaluation unit 54 evaluates a health risk using examination values pertaining to animals with the same or similar breed data stored in the storage unit 53 in addition to the examination values pertaining to the specific animal as the evaluation target by collating the breed data pertaining to the specific animal as the evaluation target with the breed data stored in the storage unit 53. A range of similarity to breed data can be predetermined at least one of by genetic information pertaining to the breed, empirically, or the like.

Specifically, the risk evaluation unit 54 evaluates, as the health risk, a probability that the specific animal is affected with a specific disease or at least one of a degree of seriousness, a lifetime, or a medical expense in a case where the specific animal is affected with the specific disease. Furthermore, the risk evaluation unit 54 evaluates the health risk when a specific time elapses (for example, X years later or the like) from the present at which evaluation is performed. The specific disease is a part or all of diseases with which the animal as the affected animal is likely to be affected, and is, for example, susceptible diseases to which the animal of the breed is susceptible empirically due to a genetic factor or the like. The probability that the specific animal is affected with the specific disease is a numerical value representing a possibility that the specific animal is affected with the specific disease, an evaluation classification regarding whether there is a high or low possibility that the specific animal is affected with the specific disease, or the like. The degree of seriousness in a case where the specific animal is affected with the specific disease, a numerical value representing slightness and seriousness of a symptom due to the affected specific disease, an evaluation classification regarding whether a possibility that the specific disease becomes serious is high or a possibility that the specific disease results in slight disease is high, or the like. The lifetime as the health risk includes a numerical value or an evaluation classification representing the length of a period (the number of years, the number of months, or the like) during which the specific animal is predicted to be alive or a numerical value or an evaluation classification representing a possibility that the specific animal is alive (or has died) at a specific point of time in the future. The medical expense as the health risk is a medical expense that is predicted to pertain to treatment or the like of the animal as the evaluation target, and is, for example, the total amount of expenses until a specific point of time in the future, an expense required for treatment or the like for affection with the specific disease, or the like.

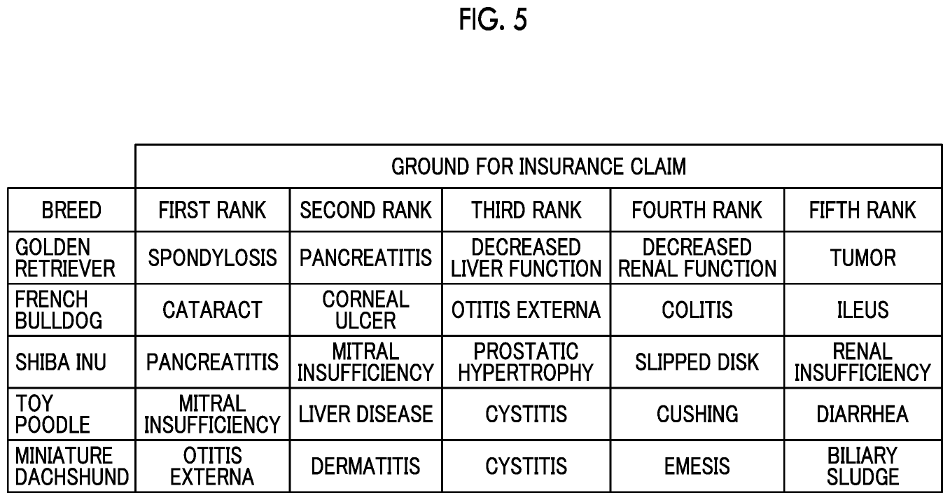

As shown in FIG. 5, even in the same animal species called dog, a ground for insurance claim is completely different depending on a breed. For this reason, the risk evaluation unit 54 decides one or a plurality of diseases (or symptoms) for evaluating a health risk using breed data. For example, in a case where the breed is Golden Retriever, since spondylosis, pancreatitis, decreased liver function, decreased renal function, tumor, and the like are susceptible disease, one or a plurality of diseases among the susceptible diseases are decided as diseases or the like for evaluating a health risk. On the other hand, in a case where the breed is French Bulldog, since cataract, corneal ulcer, otitis externa, colitis, ileus, and the like are susceptible diseases, one or a plurality of diseases among the susceptible diseases are decided as diseases for evaluating a health risk. In a case where decision is made that a health risk is evaluated on a plurality of diseases or the like, the health risk is evaluated on each of the diseases or the like. The storage unit 53 stores the above-described relationship between the breed and the susceptible disease in advance in association with each other.

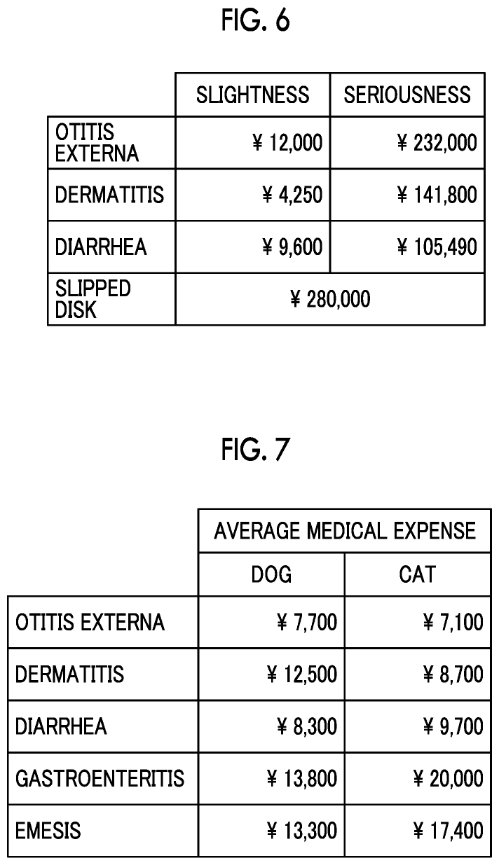

As shown in FIG. 6, even in the same disease or the like, an expense pertaining to treatment or the like is different depending on the degree of seriousness. Furthermore, as shown in FIG. 7, even in the same disease or the like, an expense pertaining to treatment or the like is different depending on the animal species. For this reason, in a case where the medical expense is evaluated as the health risk, the specific disease, the breed or the animal species, and a standard amount of money (the amount of money, such as an average medical expense shown in FIG. 6 or 7) of the medical expense are stored in the storage unit 53 in advance in association with one another, and the risk evaluation unit 54 evaluates the health risk relating to the medical expense using the stored data.

In addition to the above, the risk evaluation unit 54 evaluates a health risk by predicting transition of an examination value. The transition of the examination value is at least one of a change (increase, decrease, or the like) of the examination value, am amount of change of the examination value, deviation of the examination value from a normal value range, a deviation amount of the examination value from the normal value range, or the like. In the embodiment, a health risk is evaluated based on at least one of deviation of the examination value from the normal value range or the deviation amount of the examination value from the normal value range. The normal value range of the examination value is a value or a range of the value for determining whether the examination value is normal or abnormal, and is predetermined, for example, for each examination value by accumulation of medical knowledge, or the like. The risk evaluation unit 54 can predict transition by taking into consideration the progress or the like in which, starting from at least an examination value at an evaluation point of time of the animal as the evaluation target (a present point of time as a point of time, at which the animal visits the animal hospital 11), an animal of the same or similar breed, for which the same examination value identical or close to the examination value is obtained, follows the examination value.

The risk evaluation value provision unit 61 provides an evaluation value as an evaluation result of the health risk evaluated by the risk evaluation unit 54 to the insurance company. The provision of the evaluation value of the health risk is performed in a form in which the evaluation value of the health risk can be used for setting a plan of an animal medical insurance. That is, the risk evaluation value provision unit 61 provides the breed or the like of the animal as the evaluation target and the evaluation value of the health risk in a form capable of being associated in the insurance company 13. Furthermore, the risk evaluation value provision unit 61 can provide the evaluation value of the health risk to the insurance company 13 directly or indirectly. The direct provision of the evaluation value of the health risk means that the animal health risk evaluation system 10 is connected to a device or a system of the insurance company 13 and provides the evaluation value of the health risk. The indirect provision of the evaluation value of the health risk means that the evaluation value of the health risk is provided in a form of paper on which a list including evaluation values of one or a plurality of health risks, or the like is printed, in a form of an electronic file saved in a recording device (a flash memory or the like), or in other forms without connection to the animal health risk evaluation system 10.

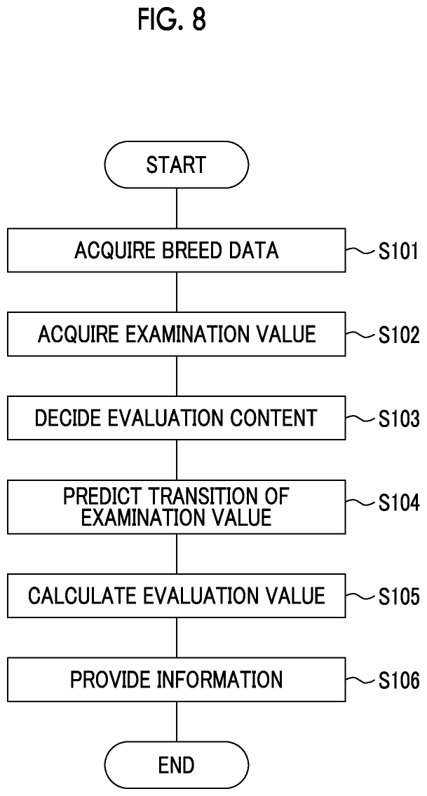

The animal health risk evaluation system 10 configured as above operates as shown in FIG. 8. First, the breed data acquisition unit 52 appropriately acquires breed data from the information terminal 17 or the like of the cooperative animal hospital 11 (Step S101). Furthermore, in a case where there are examination values that can be acquired on an animal, for which the breed data is acquired, the examination value acquisition unit 51 acquires the examination values (Step S102), and stores the examination values in the storage unit 53 in association with the breed data. Here, it is assumed that a blood examination of the animal as the evaluation target is performed, and a result of the blood examination is acquired as the examination values.

On the other hand, the risk evaluation unit 54 decides diseases or the like for evaluating a health risk using the acquired breed data (Step S103), and predicts transition of the examination values related to the diseases or the like (Step S104), thereby calculating evaluation of a health risk pertaining to each disease or the like (Step S105). Here, it is assumed that the animal as the evaluation target is Golden Retriever, and the diseases or the like for evaluating the health risk are five diseases of spondylosis, pancreatitis, decreased liver function, decreased renal function, and tumor.

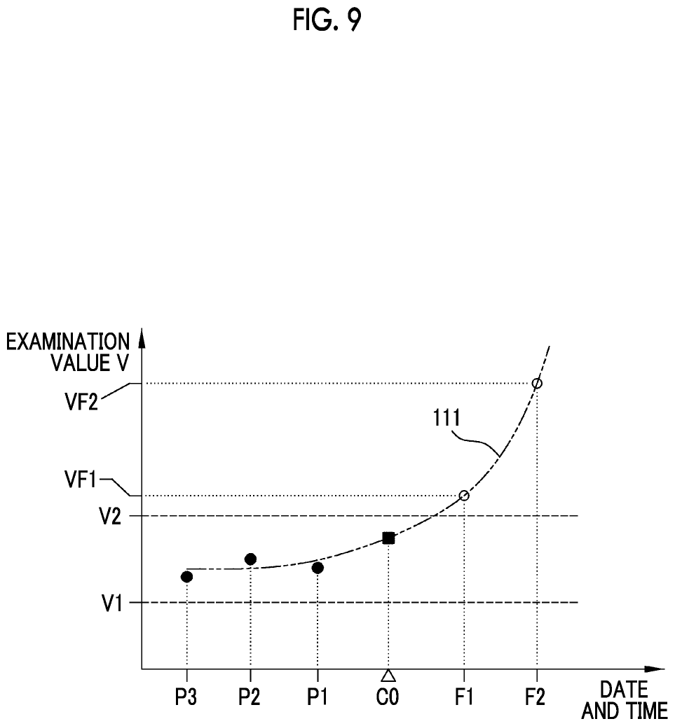

Furthermore, as shown in FIG. 9, using a specific examination value V (for example, a value of a predetermined component of blood correlated with a liver function; indicated by “▪”) on the animal as the evaluation target acquired at a present time C0 (“Δ”) at which evaluation is performed, and in a case where there is data, the same examination values V (“●”) on an animal of the same or similar breed acquired at specific points of time P1, P2, and P3 in the past and transition (or distribution) of the examination values, a curve (hereinafter, referred to as a prediction curve) 111 representing future prediction transition of the same examination value V of the animal as the evaluation target is estimated, for example, by curve fitting or the like. Then, for example, estimation values (“○”) of the same examination value V at a point of time F1 one year later and a point of time F2 two years later are obtained using the estimated prediction curve 111. Here, the estimation value of the examination value at the point of time F1 one year later is VF1, and the estimation value of the examination value at the point of time F2 two years later is VF2. Furthermore, a normal value range of the examination value V is equal to or greater than V1 and equal to or less than V2, and both of the estimation value VF1 of the examination value V one year later and the estimation value VF2 of the examination value V two years later exceed the normal value range V2.

The risk evaluation unit 54 performs the above-described estimation on one or a plurality of examination values necessary for evaluation, and calculates the evaluation value of the health risk in comprehensive consideration of the estimation values of the examination values. For example, in a case where estimation values of AST and ALT as one of items of the blood examination one year later exceed a normal value range, a probability that a symptom “decreased liver function” appears is obtained according to an excess or the like by the prediction. The same applies to “spondylosis”, “pancreatitis”, “decreased renal function”, and “tumor”, and the estimation values are obtained on one or a plurality of examination values necessary for evaluating the health risk of each disease on the specific point of time in the future. Then, the evaluation value as the health risk of each disease or the like is obtained in comprehensive consideration of the estimation values of the examination values.

Since the evaluation value of the health risk calculated as described above takes the breed of the animal as the evaluation target into consideration, the evaluation value of the health risk can be obtained for each breed of the animal, and likelihood is high. That is, the animal health risk evaluation system 10 can appropriately evaluate a risk pertaining to health of an animal.

Furthermore, in a case where the risk evaluation value provision unit 61 appropriately provides information to the insurance company 13 on appropriate evaluation of the health risk as described above (Step S106), the insurance company 13 can set at least one of an appropriate insurance plan or a special contract (option) according to the breed of the animal using the risk evaluation value. As a result, the animal as the affected animal and the keeper of the animal take at least one of the appropriate insurance plan or the special contract, whereby the keeper easily make the animal as the affected animal receive appropriate medical care on time. Furthermore, in a case where an aged Golden Retriever at 13 years old or more has a past medical history of spondylosis, pancreatitis, decreased liver function, decreased renal function, tumor, and the like, subscription to a conventional medical insurance plan is rejected. However, in a case where the evaluation value of the health risk provided by the animal health risk evaluation system 10 indicates that a possibility of affection with the susceptible diseases is low, subscription to the medical insurance plan can be accepted. Furthermore, in a case where an insurance plan based on the evaluation of the health risk is set, since subscription to the animal medical insurance is promoted, the insurance company 13 obtains the benefit of improving sales.

In the above-described first embodiment, although the risk evaluation value provision unit 61 provides the evaluation value of the health risk to the insurance company 13, the risk evaluation value provision unit 61 can provide the evaluation value of the health risk to the animal hospital 11 that cooperates with the animal health risk evaluation system 10. In a case where the evaluation value of the health risk is provided to the animal hospital 11, the veterinarian can recommend the keeper of the animal as the affected animal subscription to an appropriate animal medical insurance plan or the like according to the breed provided by the insurance company 13 based on the evaluation of the health risk. Furthermore, in a case where there is a condition (additional examination or the like) for subscription, the veterinarian can inform the keeper of the animal as the affected animal of the effect and can prompt execution of the additional examination, or the like. As a result, since the keeper of the animal can make subscription (including study) to the appropriate animal medical insurance plan with no problems, the keeper can make the animal as the affected animal receive appropriate medical care on time without feeling anxiety to a medical expense.

### Second Embodiment

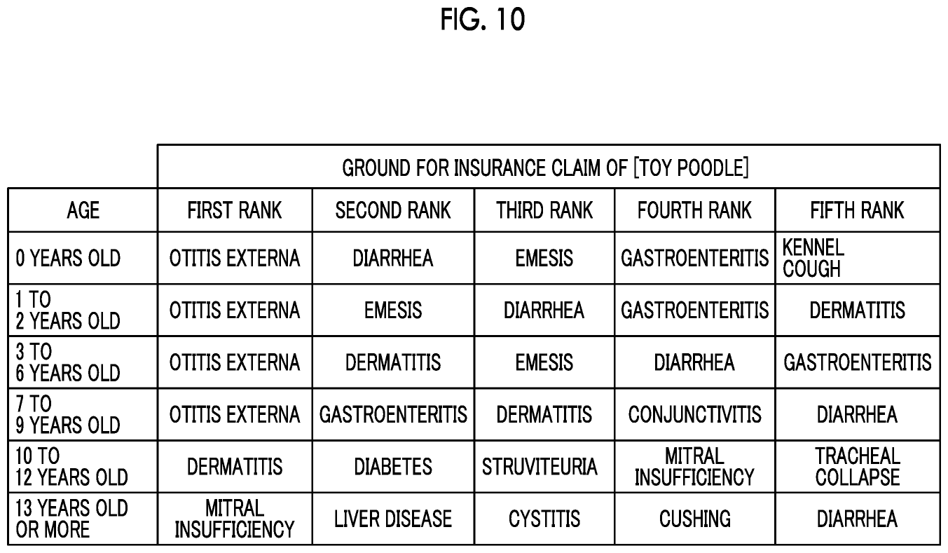

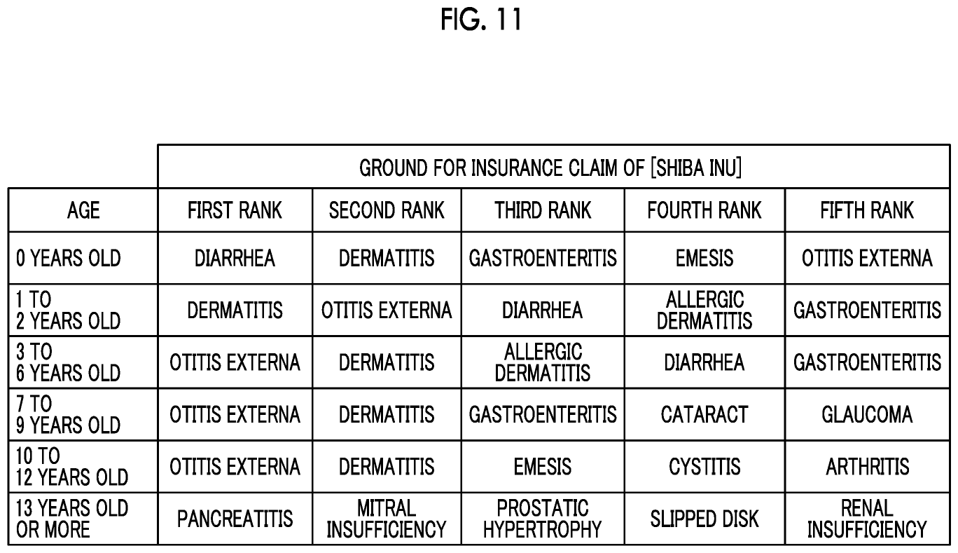

In the above-described first embodiment, although the evaluation of the health risk according to the breed of the animal as the evaluation target is performed, it is preferable to take the age of the animal as the evaluation target into consideration. This is because not only the susceptible diseases are different depending on the breed of the animal, but also as shown in FIGS. 10 and 11, the susceptible diseases are different depending on the age of the animal.

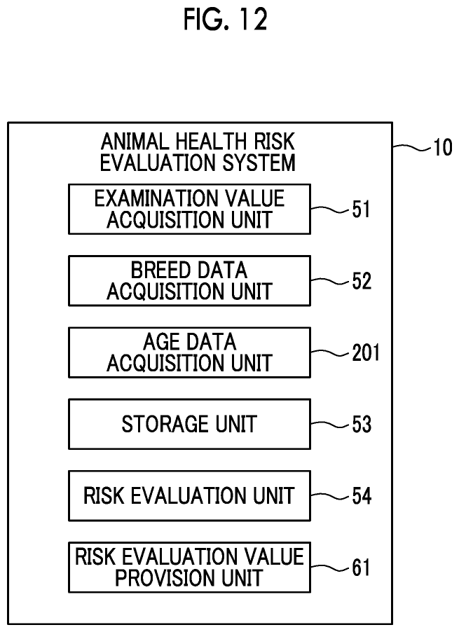

In a case where age is taken into consideration in addition to the breed in the evaluation of the health risk, as shown in FIG. 12, the animal health risk evaluation system 10 further comprises an age data acquisition unit 201. The age data acquisition unit 201 acquires age data of the animal as the evaluation target. The age data is data pertaining to age, and includes not only a numerical value representing age but also postnatal months, date of birth, or other kinds of information capable of specifying age. The age data acquisition unit 201 acquires the age data, for example, from the information terminal 17.

Furthermore, the storage unit 53 stores age in association with the breed data and the examination values. Then, the risk evaluation unit 54 evaluates a health risk of a specific animal by referring to the breed data, the examination values, and the age data stored in the storage unit 53. Specifically, the risk evaluation unit 54 specifies susceptible diseases according to the breed and the age using the breed data and the age data (see FIGS. 10 and 11). With this, the risk evaluation unit 54 decides one a plurality of diseases or the like for evaluating the health risk. Thereafter, as in the first embodiment, the risk evaluation unit 54 performs the evaluation of the health risk on the diseases or the like decided to evaluate.

As described above, in a case where the evaluation of the health risk is performed further taking age into consideration in addition to the breed of the animal, the evaluation of the health risk can be appropriately performed on the susceptible diseases that change depending on age. As a result, the animal health risk evaluation system 10 can more appropriately evaluate a health risk of an animal.

### Third Embodiment

In the first embodiment, the second embodiment, the modification examples, and the like, in a case where the insurance company 13 provides a special contract (including not only a special contract capable of being appended to a conventional insurance plan, but also a newly set insurance plan) on the animal medical insurance, information regarding the special contract is delivered from the insurance company 13 to the keeper of the animal through the animal hospital 11 or the like. For this reason, while the animal health risk evaluation system 10 cannot directly acquire the special contract on the animal medical insurance set by the insurance company 13, the animal health risk evaluation system 10 can obtain information regarding the special contract directly from the insurance company 13.

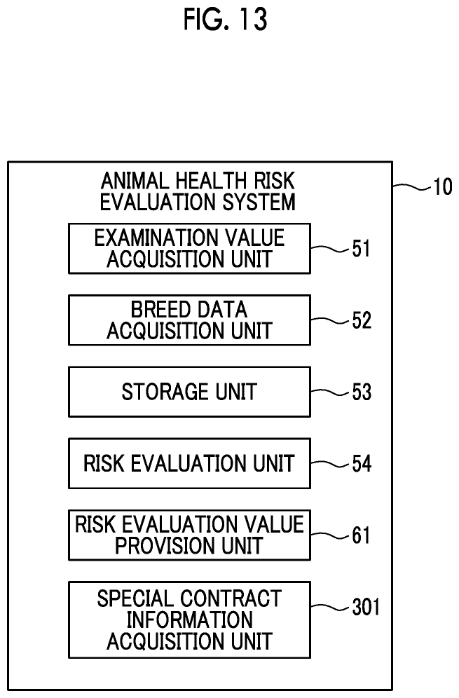

In this case, as shown in FIG. 13, the animal health risk evaluation system 10 further comprises a special contract information acquisition unit 301. In a case where the insurance company 13 that receives the evaluation value of the health risk provides a special contract on the animal medical insurance, the special contract information acquisition unit 301 acquires information (hereinafter, referred to as special contract information) pertaining to the special contract. The special contract information acquired by the special contract information acquisition unit 301 can be used for the evaluation of the health risk. For example, in a case of performing evaluation of a health risk pertaining to a medical expense, the risk evaluation unit 54 can evaluate a health risk of a real medical expense by taking the special contract information into consideration.

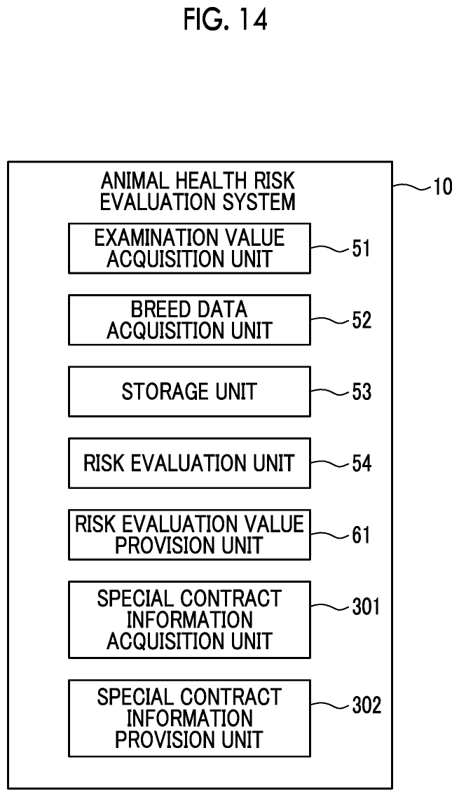

Furthermore, as shown in FIG. 14, the animal health risk evaluation system 10 can further comprise a special contract information provision unit 302. The special contract information provision unit 302 provides the special contract information to the animal hospital 11. In this way, in a case where the animal health risk evaluation system 10 provides the special contract information to the animal hospital 11 using the special contract information provision unit 302, it is possible to quickly and easily provide the special contract information to the keeper of the animal without needing distribution, close inspection, and the like of a pamphlet or the like pertaining to the animal medical insurance.

### Fourth Embodiment

In the first embodiment, the second embodiment, the third embodiment, and the modification examples, the animal health risk evaluation system 10 is a system independent of the animal hospital 11 and the insurance company 13. However, the animal health risk evaluation system 10 can be a part of a system of the animal hospital 11 or the insurance company 13. Furthermore, the units of the animal health risk evaluation system 10 may be distributed and provided in the animal hospital 11, the insurance company 13, or other independent devices or systems.

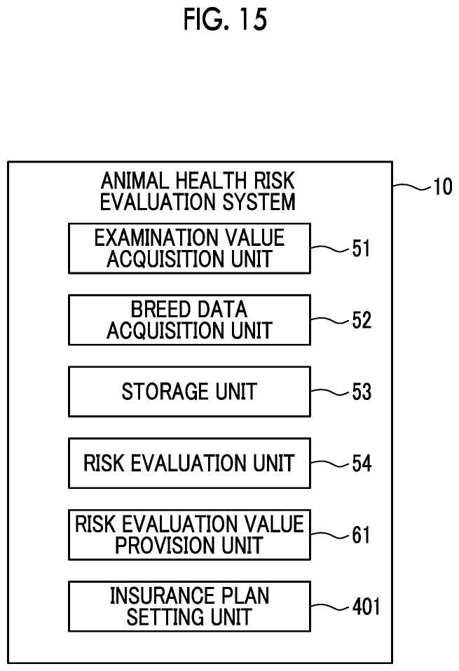

Then, in a case where the insurance company 13 holds a part or the whole of the animal health risk evaluation system 10, as shown in FIG. 15, the animal health risk evaluation system 10 can comprise an insurance plan setting unit 401. The insurance plan setting unit 401 sets at least one of a plan or a special contract pertaining to an appropriate animal medical insurance according to the breed or the like using the evaluation value of the health risk.

The animal medical insurance plan or the like set by the insurance plan setting unit 401 is, for example, a plan or a special contract that allows subscription of an animal, which is rejected to subscribe to a conventional plan since any profit cannot be made, without making an insurance premium more expensive than the conventional plan. In this case, the insurance plan setting unit 401 appropriately calculates a necessary insurance premium based on the evaluation value of the health risk.

Furthermore, in a case where there is no past medical history of the susceptible diseases, the insurance plan setting unit 401 can set a plan or a special contract to which the animal can subscribe with a premium lower than the conventional plan, for example, in a period during which the health risk is predicted to be low (a period during which the examination value is predicted not to deviate from the normal value range, or the like). In this case, the insurance plan setting unit 401 decides a premium amount or the like based on the evaluation value of the health risk, prediction transition of the evaluation value, or the like.

Furthermore, the insurance plan setting unit 401 can set a plan that pays a premium as the conventional plan in a period, during which the health risk is predicted to be low, and provides more careful protection within the period than the conventional plan (the amount of payment is greater than the conventional plan, there is a large number of kinds of diseases or the like to be a target of insurance payment, or the like). In this case, the insurance plan setting unit 401 decides the premium and the coverage based on the evaluation value of the health risk.

The first embodiment, the second embodiment, the third embodiment, the fourth embodiment, and the modification examples can be discretionarily used in combination wholly or partially.

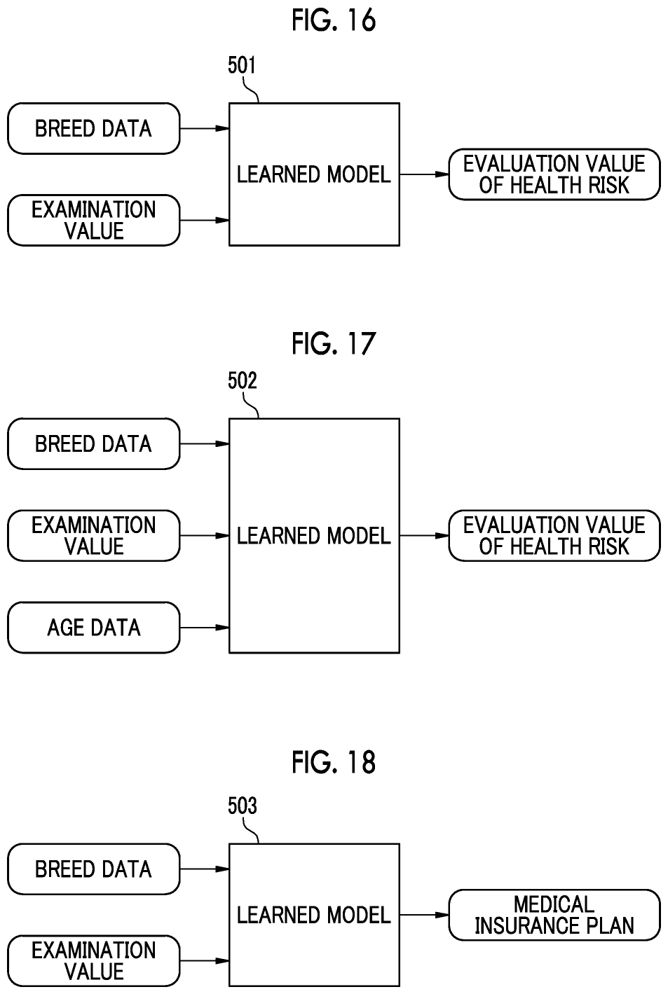

Furthermore, in the first embodiment, the second embodiment, the third embodiment, the fourth embodiment, and the modification examples, the risk evaluation unit 54 and the like can be mounted by a learned model (a so-called artificial intelligence (AI) program). For example, as shown in FIG. 16, the risk evaluation unit 54 of the first embodiment can be realized by a learned model 501 that receives an input of breed data and examination values, and outputs an evaluation value of a health risk. Furthermore, as shown in FIG. 17, the risk evaluation unit 54 of the second embodiment can be realized by a learned model 502 that receives an input of breed data, examination values, and age data, and outputs an evaluation value of a health risk. In addition, as shown in FIG. 18, the risk evaluation unit 54 and the insurance plan setting unit 401 of the fourth embodiment can be realized by a learned model 503 that receives an input of breed data and examination values, and outputs an animal medical insurance plan.

The first embodiment, the second embodiment, the third embodiment, the fourth embodiment, and the modification examples include a method of operating an animal health risk evaluation system comprising an examination value acquisition step in which the examination value acquisition unit 51 acquires an examination value as a result of an examination executed on an animal, a breed data acquisition step in which the breed data acquisition unit 52 acquires breed data as information for specifying a breed of the animal, and a risk evaluation step in which the risk evaluation unit 54 evaluates a future risk pertaining to health of a specific animal as an evaluation target using the breed data and the examination value of the specific animal. Furthermore, the embodiments include an animal health risk evaluation system in which a processor, such as a CPU, is configured to acquire an examination value as a result of an examination executed on an animal, acquire breed data as information for specifying a breed of the animal, and evaluate a future risk pertaining to health of a specific animal as an evaluation target using the breed data and the examination value of the specific animal, an animal health risk evaluation program, and an animal health risk evaluation method.

The animal health risk evaluation system 10 of the first embodiment, the second embodiment, the third embodiment, the fourth embodiment, and the modification examples can be realized by a method that causes a computer having a processor, a memory, and the like to execute processing pertaining to the above-described animal health risk evaluation method. For example, the animal health risk evaluation system 10 can be realized by causing a computer to execute processing of reading an examination value as a result of an examination executed on an animal and breed data as information for specifying a breed of the animal from a database, which stores the examination value in association with the breed data, and evaluating a future risk (health risk) pertaining to health of a specific animal as an evaluation target using the examination value and the breed data read from the database, and breed data and an examination value of the specific animal. Furthermore, the animal health risk evaluation system 10 of the fourth embodiment having the insurance plan setting unit 401 can be realized by causing a computer to execute processing of presenting at least one of an insurance plan or a special contract based on the health risk in addition to the above-described processing. In these cases, although the computer that executes the processing pertaining to the animal health risk evaluation method can have the database, which stores the examination value in association with the breed data, in the form of, for example, the storage unit 56, the database may be an external database that can be accessed by the computer.

In the above-described embodiments and the modification examples, the hardware structures of the processing units that execute various kinds of processing, such as the examination value acquisition unit 51, the breed data acquisition unit 52, the storage unit 53, the risk evaluation unit 54, the risk evaluation value provision unit 61, the age data acquisition unit 201, the special contract information acquisition unit 301, the special contract information provision unit 302, and the insurance plan setting unit 401, are various processors described below. Various processors include a central processing unit (CPU) that is a general-purpose processor executing software (program) to function as various processing units, a programmable logic device (PLD) that is a processor capable of changing a circuit configuration after manufacture, such as a graphical processing unit (GPU) or a field programmable gate array (FPGA), a dedicated electric circuit that is a processor having a circuit configuration dedicatedly designed for executing various kinds of processing, and the like.

One processing unit may be configured of one of various processors described above or may be configured of a combination of two or more processors (for example, a plurality of FPGAs, a combination of a CPU and an FPGA, or a combination of a CPU and a GPU) of the same type or different types. A plurality of processing units may be configured of one processor. As an example where a plurality of processing units are configured of one processor, first, as represented by a computer, such as a client or a server, there is a form in which one processor is configured of a combination of one or more CPUs and software, and the processor functions as a plurality of processing units. Secondly, as represented by system on chip (SoC) or the like, there is a form in which a processor that realizes all functions of a system including a plurality of processing units into one integrated circuit (IC) chip is used. In this way, various processing units are configured using one or more processors among various processors described above as a hardware structure.

In addition, the hardware structure of various processors is, more specifically, an electric circuit (circuitry), in which circuit elements, such as semiconductor elements, are combined. Furthermore, the hardware structure of the storage unit is a storage device, such as a hard disc drive (HDD) or a solid state drive (SSD).

### EXPLANATION OF REFERENCES

10: animal health risk evaluation system
11: animal hospital
11A: first animal hospital
11B: second animal hospital
11C: third animal hospital
13: insurance company
16: examination device group
17: information terminal
21: blood examination device
22: X-ray examination device
23: ultrasound examination device
24: MRI examination device
25: CT examination device
31: urine examination device
32: fecal examination device
33: eye examination device
34: oral examination device
35: skin examination device
51: examination value acquisition unit
52: breed data acquisition unit
53: storage unit
54: risk evaluation unit
61: risk evaluation value provision unit
111: prediction curve
201: age data acquisition unit
301: special contract information acquisition unit
302: special contract information provision unit
401: insurance plan setting unit
501: learned model
502: learned model
503: learned model
C0: present
F1: future point of time
F2: future point of time
P1, P2, P3: past point of time
S101 to S106: step of operation
V: examination value
V1: lower limit of normal value range
V2: upper limit of normal value range
VF1: estimation value
VF2: estimation value

What is claimed is:

1. An animal health risk evaluation system comprising:
a processor configured to function as:
an examination value acquisition unit that acquires an examination value as a result of an examination executed on an animal;
a breed data acquisition unit that acquires breed data as information for specifying a breed of the animal;
a storage unit that stores the breed data in association with the examination value; and
a risk evaluation unit that evaluates a future risk pertaining to health of a specific animal as an evaluation target using the breed data and the examination value of the specific animal.

2. The animal health risk evaluation system according to claim 1,
wherein the examination value acquisition unit acquires the examination value directly or indirectly from examination devices provided in one or a plurality of animal hospitals.

3. The animal health risk evaluation system according to claim 1,
wherein the examination value acquisition unit acquires one or a plurality of the examination values including a result of a blood examination.

4. The animal health risk evaluation system according to claim 2,
wherein the examination value acquisition unit acquires one or a plurality of the examination values including a result of a blood examination.

5. The animal health risk evaluation system according to claim 1,
wherein the risk evaluation unit evaluates, as the risk, at least one of a probability that the specific animal is affected with a specific disease, a degree of seriousness in a case where the specific animal is affected with the specific disease, a lifetime, or a medical expense.

6. The animal health risk evaluation system according to claim 2,
wherein the risk evaluation unit evaluates, as the risk, at least one of a probability that the specific animal is affected with a specific disease, a degree of seriousness in a case where the specific animal is affected with the specific disease, a lifetime, or a medical expense.

7. The animal health risk evaluation system according to claim 3,
wherein the risk evaluation unit evaluates, as the risk, at least one of a probability that the specific animal is affected with a specific disease, a degree of seriousness in a case where the specific animal is affected with the specific disease, a lifetime, or a medical expense.

8. The animal health risk evaluation system according to claim 1,
wherein the risk evaluation unit evaluates the risk when a specific time elapses from a present time at which evaluation is performed.

9. The animal health risk evaluation system according to claim 2,
wherein the risk evaluation unit evaluates the risk when a specific time elapses from a present time at which evaluation is performed.

10. The animal health risk evaluation system according to claim 3,
wherein the risk evaluation unit evaluates the risk when a specific time elapses from a present time at which evaluation is performed.

11. The animal health risk evaluation system according to claim 5,
wherein the risk evaluation unit evaluates the risk when a specific time elapses from a present time at which evaluation is performed.

12. The animal health risk evaluation system according to claim 1,
wherein the risk evaluation unit evaluates the risk by predicting transition of the examination value.

13. The animal health risk evaluation system according to claim 12,
wherein the risk evaluation unit evaluates the risk based on at least one of deviation of the examination value from a normal value range or a deviation amount of the examination value from the normal value range.

14. The animal health risk evaluation system according to claim 1,
wherein the processor is further configured to function as an age data acquisition unit that acquires age data of the animal, and
the storage unit stores the age data in association with the breed data and the examination value, and the risk evaluation unit evaluates the risk of the specific animal by referring to the breed data, the examination value, and the age data stored in the storage unit.

15. The animal health risk evaluation system according to claim 1,
wherein the processor is further configured to function as a risk evaluation value provision unit that provides an evaluation value of the risk evaluated by the risk evaluation unit to an insurance company.

16. The animal health risk evaluation system according to claim 15,
wherein the processor is further configured to function as a special contract information acquisition unit that, in a case where the insurance company that receives the evaluation value of the risk provides a special contract on an animal medical insurance, acquires information pertaining to the special contract.

17. The animal health risk evaluation system according to claim 16,
wherein the processor is further configured to function as a special contract information provision unit that provides the information pertaining to the special contract to an animal hospital.

18. An animal health risk evaluation method that causes a computer to execute processing of reading an examination value as a result of an examination executed on an animal and breed data as information for specifying a breed of the animal from a database, which stores the examination value in association with the breed data, and evaluating a future risk pertaining to health of a specific animal as an evaluation target using the examination value and the breed data read from the database, and the breed data and the examination value of the specific animal.

19. The animal health risk evaluation method according to claim 18, causing the computer to execute processing of presenting at least one of an insurance plan or a special contract based on the risk.
